(19) United States
(12) Patent Application Publication
(10) Pub. No.: US 2023/0051524 A1
(43) Pub. Date: Feb. 16, 2023

(54) A CHICKEN METHYLATION CLOCK

(71) Applicant: Evonik Operations GmbH, Essen (DE)

(72) Inventors: Florian BOEHL, Neckargemuend (DE); Frank LYKO, Hirschberg an der Bergstrasse (DE); Rose WHELAN, Ambrosden (GB); Andreas KAPPEL, Glashuetten (DE); Emeka Ignatius IGWE, Muenchen (DE); Frank THIEMANN, Nottuln (DE); Guenter RADDATZ, Heidelberg (DE)

(73) Assignee: Evonik Operations GmbH, Essen (DE)

(21) Appl. No.: 17/784,525

(22) PCT Filed: Dec. 8, 2020
(86) PCT No.: PCT/EP2020/085085

§ 371 Date: Jun. 10, 2022

(30) Foreign Application Priority Data
Dec. 13, 2019 (WO) PCT/EP2019/085021

(51) Int. Cl. C12Q 1/6883

(52) U.S. Cl. CPC C12Q 1/6883; C12Q 2600/112; C12Q 2600/124; C12Q 2600/154

(57) ABSTRACT

The invention provides a method of establishing a chicken methylation clock comprising: (a) determining the methylation ratio and the read coverage of the genomic CpG sites of an age-correlated training sample of a specific chicken tissue; (b) defining a set of CpG sites having reliable methylation ratios in all training samples of step (a) using a cutoff value; and (c) performing a penalized regression using the methylation ratios of step (b) as input and the age correlated to the training sample as dependent variable, by applying a penalized regression model; thereby obtaining a set of CpG sites with corresponding weighting factors and intercept of the linear model equation as parameters defining the chicken methylation clock.

### FIELD OF THE INVENTION

The present invention relates to a method for determining the chronological and epigenetic age of chicken (“chicken methylation clock”).

### BACKGROUND OF THE INVENTION

The chicken (Gallus gallus) is an important non-mammalian vertebrate model organism and a significant source of commercially produced meat and eggs. Factors that influence the growth, pathogen resistance and meat quality of chicken are thus of considerable scientific and economical interest. Extensive genome-wide association studies have been conducted to elucidate the underlying genetic framework. Epigenetic modifications provide an important complement and extension to genetic variants but have remained relatively underexplored in chicken.

Animal methylomes can be highly diverse, ranging from certain insect genomes with sparse methylation patterns and only tens of thousands of methylation marks to mammalian genomes with dense methylation patterns and tens of millions of methylation marks. Until now, only little is known about the genome-wide DNA methylation patterns of non-mammalian vertebrates, and particularly of birds.

DNA methylation correlates with ageing processes and represents an epigenetic modification with a high specificity for CpG dinucleotides (5'—C—phosphate—G—3'), i.e. regions of DNA where a cytosine nucleotide is followed by a guanine nucleotide in the linear sequence of bases along its 5' → 3' direction.

Lowly-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described in the mouse (Stadler et al. Nature 480, 490-495 (2011)). Evolutionary conservation of LMRs beyond mammals has remained unexplored.

Age-correlated DNA methylation changes at discrete sets of CpGs in the human genome have been identified and used to predict age (Horvath, S. (2013). DNA methylation age of human tissues and cell types. Genome Biology 14:3156). These “epigenetic clocks” can estimate the DNA methylation age in specific tissues or tissue-independently and can predict mortality and time to death.

Epigenetic age is highly correlated with chronological age but can, depending on environmental factors accelerating or decelerating ageing processes, deviate substantially from chronological age.

Epigenetic age acceleration (epigenetic age > chronological age) suggests that the underlying tissue ages faster than expected on the basis of chronological age, whereas a negative value (epigenetic age < chronological age, age deceleration) suggests that the tissue ages slower than would be expected. Epigenetic age acceleration is associated with a great number of age-related conditions and diseases, such as e.g. inflammatory processes in the gut.

When it comes to welfare and performance of livestock chickens, intestinal health is critically important. Enteric diseases, which are usually associated with inflammatory processes and affect the structural integrity of the gastrointestinal tract (GIT) lead to high economic losses due to reduced weight gain, poor feed conversion efficiency, increased mortality rates and greater medication costs (M’Sadeq, S.A., Wu, S., Swick, R.A. & Choct, M. (2015). Towards the control of necrotic enteritis in broiler chickens with in-feed antibiotics phasing-out worldwide. Animal Nutrition, 1, 1-11; Timbermont, L., Haesebrouck, F., Ducatelle, R. & Van Immerseel, F. (2011). Necrotic enteritis in broilers: an updated review on the pathogenesis. Avian Pathol, 40, 341-347).

Accordingly, new descriptive and predictive markers for biological conditions (such as inflammation of the gut) are urgently needed for controlling ongoing production processes and enabling early intervention, where necessary.

In view of the above, it was the objective of the invention to develop methods for determining the chronological age of chicken (“chicken methylation clock”), or for determining age acceleration in chicken, that are also suitable for evaluating and/or monitoring the health condition of a chicken flock.

### SUMMARY OF THE INVENTION

The aforementioned objectives are solved by the different embodiments of the present invention. Generally, and by way of brief description, the main aspects of the present invention can be described as follows:

In a first aspect, the invention pertains to a method of establishing a chicken methylation clock, the method comprising:
(a) determining the methylation ratio and the read coverage of the genomic CpG sites of an age-correlated training sample of a specific chicken tissue;
(b) defining a set of CpG sites having reliable methylation ratios in all training samples of step (a) using a cutoff value; and
(c) performing a penalized regression using the methylation ratios of step (b) as input and the age correlated to the training sample as dependent variable, by applying a penalized regression model;
thereby obtaining a set of CpG sites with corresponding weighting factors and intercept of the linear model equation as parameters defining the chicken methylation clock.

In a second aspect, the invention provides an in vitro method for predicting the chronological age of chicken, the method comprising the steps of:
a) obtaining genomic chicken DNA from biological sample material deriving from the chicken subject or from the chicken population to be tested;
b) determining the methylation level of for the CpG sites indicated in Table 2 or, alternatively, for the CpG sites indicated in Table 3, in the genomic chicken DNA;
c) comparing the methylation levels of these CpG sites in the genomic chicken DNA from the sample to be tested with the methylation levels of the same CpG sites from an age-correlated reference sample,
and deducing therefrom the chronological age of the subject or the population to be tested.

In a specific embodiment of the present invention, the set of specific CpG sites corresponds to the set of 63 methylation markers listed in Table 2 below (genome-wide methylation approach).

In a specific embodiment of the present invention, the set of specific CpG sites corresponds to the set of 54 LMR-associated methylation markers listed in Table 3 below (LMR methylation approach).

In a third aspect, the invention relates to a method for predicting the chronological age of a chicken tissue sample, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain the weighted methylation ratios of those CpG sites, (c) computing the sum over the weighted methylation ratios obtained in step (b) and adding the respective intercept of linear model equation,
thus predicting a chronological age for the chicken tissue sample.

In a fourth aspect, the invention the invention is directed to a method for detecting accelerated aging in chicken tissue samples, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain the weighted methylation ratios of those CpG sites,
(c) computing the sum over the weighted methylation ratios obtained in step (b) and adding the respective intercept of linear model equation, thus predicting the age for the chicken tissue sample (epigenetic age) and
(d) comparing the predicted age of step (c) with the actual chronological age of the tissue sample,
wherein a predicted age higher than the chronological age is indicative of accelerated aging in the chicken tissue.

As epigenetic age acceleration (i.e. epigenetic age > chronological age) is correlated with environmental responses (e.g. imbalance of the gut microbiome), specific conditions or disorders, such as inflammation of the gut, the above method serves as a diagnostic tool that is particularly suitable for evaluating and/or monitoring the health condition of a chicken flock.

### DETAILED DESCRIPTION OF THE INVENTION

In the following, the elements of the invention will be described. These elements are listed with specific embodiments; however, it should be understood, that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that the application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

The term “test” / “tested” shall in the present disclosure indicate that an entity is subjected to an analysis application of the present invention.

The term “genomic DNA” shall refer to DNA molecules or fragments of the genome of the subject or group of subjects.

In context of the present invention, the terms “methylation profile”, “methylation pattern”, “methylation state” or “methylation status,” are used herein to describe the state of methylation of a genomic sequence, and such term refers to the characteristics of a DNA segment at a particular genomic locus relevant to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated). Thus, based on the methylation status of one or more methylation sites, a methylation profile may be determined. Accordingly, the term "methylation" profile" or also "methylation pattern" refers to the relative or absolute concentration of methylated C or unmethylated C at any particular stretch of residues in a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., sample nucleic acid) are methylated as compared to another sequence from a different region or from a different individual (e.g., relative to normal nucleic acid), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated.

As used herein, a “CpG site” or “methylation site” is a nucleotide within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

The term “bisulfite” as used herein encompasses any suitable type of bisulfite, such as sodium bisulfite, or other chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

The methylation age depends on the biological state or condition of an individual or of a population and takes into account the circumstances of life (such as stress, nutrition, etc.).The terms “methylation age”, “epigenetic age”, and “biological age” have identical meanings and are used interchangeably in the context of the present application.

The term “methylation marker”, “clock CpG” or “CpG site” as used in the context of the present invention refers to a CpG position that is potentially methylated. Methylation typically occurs in a CpG containing nucleic acid. The CpG containing nucleic acid may be present in, e.g. a CpG island, a CpG doublet, a promoter, an intron, or an exon of a gene. For instance, the potential methylation sites may encompass the promoter/enhancer regions of the indicated genes. The “set of specific CpG sites in the genomic chicken DNA” refers to the CpG sites showing the best correlations with age.

As used in the context of the present invention, the term “chicken” refers to the species Gallus gallus.

### Establishing a Chicken Methylation Clock

The inventors have developed a method of establishing a chicken methylation clock. This method comprises
(a) determining the methylation ratio and the read coverage of the genomic CpG sites of an age-correlated training sample of a specific chicken tissue;
(b) defining a set of CpG sites having reliable methylation ratios in all training samples of step (a) using a cutoff value; and
(c) performing a penalized regression using the methylation ratios of step (b) as input and the age correlated to the training sample as dependent variable, by applying a penalized regression model;
thereby obtaining a set of CpG sites with corresponding weighting factors and intercept of the linear model equation as parameters defining the chicken methylation clock.

Based on the above findings, the inventors have identified a number of CpG (Cytosine-phosphate-Guanine) sites in the chicken (Gallus gallus) genome for which the level of DNA methylation is both tissue-specifically and tissue-independently correlated with chronological age.

That is, measuring DNA methylation at these locations (CpG sites) enables making accurate predictions of the chronological age of chicken, respectively.

The term “methylation ratio” refers to number of methylated cytosine(s) divided by the total number of cytosine(s) covered at the specific site(s)

The term “read coverage of the CpG site” is to be understood as the number of reads that align with the known CpG site in the reference sequence.

The methylation ratio and the read coverage of the genomic CpG sites may be determined in step (a) using bisulfite sequencing.

The age-correlated training sample of a specific tissue used in step (a) may, for example, be gut tissue, muscle tissue, organ tissue or skin tissue.

Preferably, the coverage cutoff value defined in step (b) is 3.

The method described above may further comprise a step of (d) optimizing the fit of the regression model such that the number of CpG sites is reduced to below 100. For optimizing the fit of the regression model in step (d), the algorithm preferably applies ridge regression in combination with lasso regression. Advantageously, the methods of ridge regression and lasso regression are balanced using an alpha value of between 0 and 1.

### Prediction of Chronological Age

Based on the above, the inventors have developed multi-tissue age predictors for chicken (“chicken methylation clocks”). These multi-tissue age predictors are widely applicable, as for most tissues and it does not require any adjustments or offsets.

The term “chronological age” refers to the calendar time that has passed from birth/hatch.

One specific embodiment of the present invention pertains to an in vitro method for predicting the chronological age of chicken, the method comprising the steps of:
a) obtaining genomic chicken DNA from biological sample material deriving from the chicken subject or from the chicken population to be tested;
b) determining the methylation level of for the CpG sites indicated in Table 2 (based on a genome-wide methylation approach) or, alternatively, for the CpG sites indicated in Table 3 (based on an LMR based methylation approach), in the genomic chicken DNA;
c) comparing the methylation levels of these CpG sites in the genomic chicken DNA from the sample to be tested with the methylation levels of the same CpG sites from an age-correlated reference sample, and deducing therefrom the chronological age of the subject or the population to be tested.

The age-correlated reference sample serves as a control and represents an average methylation level at a pre-determined and specific chronological age.

The method for predicting the chronological age of chicken can be used for testing individual animals and for testing a complete animal population, such as a chicken or broiler/layer flock.

The biological sample material deriving from the subject or from the population to be tested is selected from the group consisting of body fluids; excremental material; tissue material, such as muscle tissue, gut tissue, organ tissue, skin tissue; feather material, such as quill pen; or combinations thereof. Examples for body fluids are blood and sailva. Excremental material includes gut content, fecal and cecal excrements, as well as mixtures, solutions or suspensions thereof. An example for muscle tissue is breast (pectoralis major), examples for gut tissue are ileum and jejunum; and examples for organ tissue are spleen tissue or heart tissue.

Step b) according to the present invention may include a bisulfite conversion process. In step c) a regression analysis may be used determine the epigenetic age of the subject or the population tested. Details on the bisulfite conversion and regression analysis are provided in the definitions above and in the experimental section below.

One specific embodiment of the present invention pertains to method for predicting the chronological age of a chicken tissue sample, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain the weighted methylation ratios of those CpG sites, (c) computing the sum over the weighted methylation ratios obtained in step (b) and adding the respective intercept of linear model equation,
thus predicting the chronological age for the chicken tissue sample.

The tissue sample material deriving from a chicken subject or from a chicken population to be tested used in step (a) may be selected from the group consisting of gut tissue, muscle tissue, organ tissue or skin tissue. Preferably, the tissue sample material deriving from a chicken subject or from a chicken population to be tested used in step (a) is gut tissue, preferably isolated from fecal sample material.

The methylation ratios for the CpG sites in step (b) were advantageously be determined using bisulfite sequencing.

### Detecting Accelerated Aging

The epigenetic age depends on the biological state or condition of an individual (or of a population).

Epigenetic age may match or mismatch with chronological age. Deviations of the epigenetic age from the chronological age are age acceleration or age deceleration.

Accordingly, epigenetic age may also be determined by comparison of the methylation levels of the methylation markers (i.e. CpG sites) in the genomic chicken DNA from the sample to be tested with the methylation status of the same markers (i.e. CpG sites) from an age-correlated reference sample. The term “age-correlated reference sample” is to be understood as defined above.

More specifically, the present invention in one embodiment provides an in vitro method for determining or establishing the epigenetic age of chicken, the method comprising the steps of: (a) obtaining genomic chicken DNA from biological sample material deriving from the subject or from the population to be tested; (b) determining the methylation level of a set of specific CpG (5'-Cytosine-phosphoguanine) sites (“clock CpGs”) in the genomic chicken DNA; and c) comparing the methylation levels of these CpG sites in the genomic chicken DNA from the sample to be tested with the methylation status of the same CpG sites from an age-correlated reference sample (“control”); thereby establishing the epigenetic age of the subject or the population to be tested.

The biological sample material deriving from the subject or from the population to be tested is selected from the group consisting of body fluids; excremental material; tissue material, such as muscle tissue, gut tissue, organ tissue, skin tissue; feather material, or combinations thereof. Examples for body fluids and tissue material are provided in the above.

The excremental sample material may be selected from the group consisting of gut content, litter samples and samples of bodily excrements and solutions or suspensions thereof. The term “litter sample” refers to mixed fecal droppings comprising residues of bedding material.

The biological sample deriving from the subject or from the population to be tested is preferably feces. Fecal sample material can be collected ante mortem. The DNA material isolated from feces contains significant amounts of gut cell DNA (mucosa).

In a particularly preferred embodiment, biological sample deriving from the subject or from the population to be tested is pooled fecal sample material deriving from a chicken population. Pooled fecal sample material is obtained by combining and mixing individual fecal samples.

The sample size (i.e. the number of excremental samples to be taken; each sample taken at a specific site within the animal house) has to be determined in view of the actual stocking density, i.e. with the actual number of animals belonging to the population to be tested.

In general, a minimum of 80 to 100 individual excremental samples are sufficient for most livestock chicken populations. As an example, for a broiler flock of 20000 animals, 96 individual samples are required for a confidence level of 95%.

For obtaining the pooled excremental sample material, several sampling methods may be used. In one embodiment, the pooled excremental sample is obtained by systematic grid sampling (systematic random sampling). For this method, the animal house or area in which the avian population is kept is divided in a grid pattern of uniform cells or sub-areas based on the desired number of individual excremental samples (i.e. the sample size). Then, a random sample collection site is identified within the first grid cell and a first sample is taken at said site. Finally, further samples are obtained from adjacent cells sequentially - e.g. in a serpentine, angular or zig-zag fashion - using the same relative location within each cell. A random starting point can be obtained with a dice or a random number generator. The above process may optionally be repeated for replicate samples.

As an example for broiler flocks, excremental samples may be collected and analyzed on a daily basis during the initial growth phase (starter phase, day 5 to day 10), and/or during the enhanced growth phase (day 11 to day 18) and, optionally, also on a later stage. Alternatively, the excremental sample material, in particular fecal sample material, from the broiler flock is collected and analyzed on a daily basis starting from day 10.

Step (b), determining the methylation level of a set of specific CpG (5'-Cytosine-phosphoguanine) sites (“clock CpGs”) in the genomic chicken DNA, may include a bisulfite conversion process. Therein, cytosine residues in the genomic DNA are transformed to uracil, while 5'-methylcytosine residues in the genomic DNA are not transformed to uracil.

Whole genome bisulfite sequencing is a genome-wide analysis of DNA methylation based on the sodium bisulfite conversion of genomic DNA, which is then sequenced on a next-generation sequencing platform. The sequences are then re-aligned to the reference genome to determine methylation states of the CpG dinucleotides based on mismatches resulting from the conversion of unmethylated cytosines into uracil.

For example, methylation levels can be measured using the commercial Illumina™ platform.

To quantify the methylation level, various established protocols may be used to calculate the beta value of methylation, which equals the fraction of methylated cytosines in a specific location.

Step c) may be performed with a statistical prediction method. Preferably, a regression analysis, such as penalized regression is used in step c) to determine the epigenetic age of the subject or the population tested.

In one embodiment of the determining the epigenetic age of chicken according to the present invention, the set of methylation markers (i.e. CpG sites) corresponds to the 63 CpG sites indicated in Table 2 below (genome-wide methylation approach). In an alternative embodiment, the set of methylation markers (i.e. CpG sites) corresponds to the 54 CpG sites indicated in Table 3 below (LMR methylation approach).

The invention also provides a method for detecting accelerated aging in chicken tissue samples, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain the weighted methylation ratios of those CpG sites,
(c) computing the sum over the weighted methylation ratios obtained in step (b) and adding the respective intercept of linear model equation, thus predicting the age for the chicken tissue sample (epigenetic age) and
(d) comparing the predicted age of step (c) with the actual chronological age of the tissue sample,
wherein a predicted age higher than the chronological age is indicative of accelerated aging in the chicken tissue.

The methods described above are applicable for individual subjects, i.e. an individual chicken, and also for a whole chicken population, such as a flock of broilers or layers.

The life cycle of chicken starts with eggs taken from parent birds in the hatchery which are then incubated at a constant temperature for 21 days until the birds hatch, though at this stage the precocial chicken might be up to 72 hours old they are called one-day chicken. These chickens are separated by sexes and the female birds are kept for approx. one year for laying eggs.

The life span for broiler chicken is significantly shorter and varies between 21 days up to 170 days. An average US broiler is slaughtered after 47 days at a slaughter weight of 2.6 kg while in Europe the average slaughter age is at 42 days (at a weight of 2.5 kg).

Broilers are usually kept in flocks which can consist of 20.000 birds or more in one house and are fed with up to three different feed types (starter feed, grower feed and finisher feed) during this production cycle.

The birds are usually exposed to a number of external environmental factors, such as bacteria, viruses, parasites, diet or climate. These factors influence the outcome of a production cycle in terms of flock performance or flock uniformity and manifest in a different methylation pattern of a single bird or of a flock which may result in age acceleration that could be detected.

The inventors have found that in chicken, a mismatch of epigenetic and chronological age, and in particular epigenetic age acceleration (i.e. epigenetic age > chronological age) is an early indication for sub-optimal health condition, caused by clinical or sub-clinical/latent conditions or disorders.

The methods according to the present invention may thus be used for monitoring the health condition of an individual or of a population over time and/or for determining the health condition of a chicken livestock, and, accordingly, for determining the necessity of therapeutic or nutritional interventions.

Accordingly, the method may include providing an individualized (tailored) treatment to the individual or population tested to bring the predicted epigenetic age closer to the chronological age of the individual or population.

Such treatment or intervention may include feeding or administering health-promoting substances, such as zootechnical feed additives, or therapeutic agents. The term “administering” or related terms includes oral administration. Oral administration may be via drinking water, oral gavage, aerosol spray or animal feed. The term “zootechnical feed additive” refers to any additive used to affect favorably the performance of animals in good health or used to affect favorably the environment. Examples for zootechnical feed additives are digestibility enhancers, i.e. substances which, when fed to animals, increase the digestibility of the diet, through action on target feed materials; gut flora stabilizers; micro-organisms or other chemically defined substances, which, when fed to animals, have a positive effect on the gut flora; or substances which favorably affect the environment. Preferably, the health-promoting substances are selected from the group consisting of probiotic agents, prebiotic agents, botanicals, organic/fatty acids, bacteriophages and bacteriolytic enzymes or any combinations thereof.

In addition to the above, the present invention also pertains to the use of the methods disclosed herein for the development of a routine analysis tool such as real-time PCR, targeted sequencing/panel sequencing, methylated DNA immunoprecipitation as input for both, chip/array technology or methylated DNA sequencing.

Finally, the present invention provides tangible computer-readable medium comprising a computer readable code that, when executed by a computer, causes the computer to perform operations comprising: a) receiving information corresponding to methylation levels of a set of CpG sites (i..e. methylation markers; “clock CpGs”) in the genomic chicken DNA in a biological sample deriving from a subject or from a population to be tested; and b) determining the epigenetic age of the subject or the population to be tested by applying a statistical prediction algorithm to the measured methylation levels of these CpG sites, wherein the set of CpG sites comprises the CpG sites indicated in Table 2, or the CpG sites indicated in Table 3.

Applications of the methods according to the invention are for example ((i) aiding in evaluation of the health status of chicken (ii) monitoring the progress or reoccurrence of clinical and sub-clinical disorders or (iii) studying the effects of medication, feed compounds and/or special diets on the biological age - and thus on the health status of chicken.

Applications of the methods according to the present invention in particular help to avoid loss in animal performance like weight gain and feed conversion.

### BRIEF DESCRIPTION OF THE FIGURES

FIG. 1. Mean squared error of a trained clock for given alpha at value of lambda leading to the minimal error.

FIG. 2. Number of CpGs for given alpha at value of lambda leading to the minimal error.

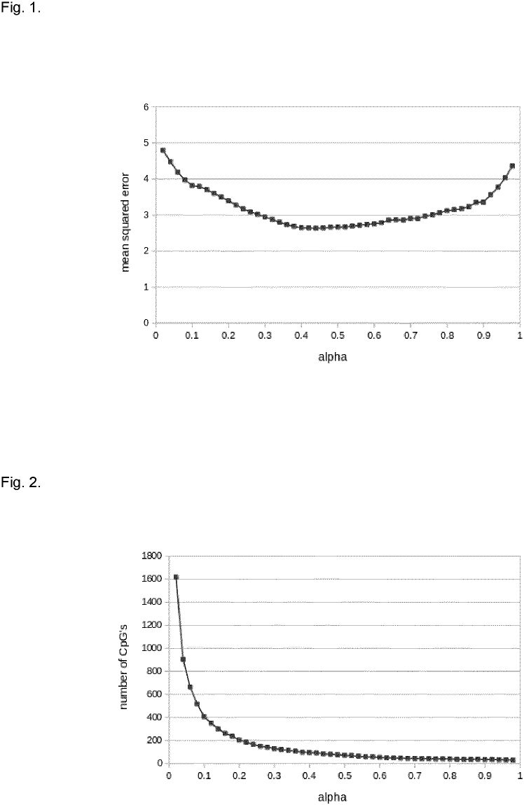

### EXAMPLES

### Methods

A broiler study was conducted with Ross 308 male broilers fed industry standard, three phase, corn-soybean meal diets formulated to meet all nutrient requirements from day 1-35 (Table 1).

Table 1

| Ingredients, % | Starter (day 1-14) | Grower (day 15 - 28) | Finisher (day 29 -35) |
|---|---|---|---|
| Corn | 54.38 | 62.93 | 63.24 |
| Soybean Meal (48% CP) | 35.00 | 26.83 | 25.48 |
| Corn Gluten Meal (60% CP) | 4.00 | 4.00 | 4.00 |
| Soybean Oil | 2.66 | 2.45 | 3.80 |
| Dicalcium phosphate 22 | 1.74 | 1.61 | 1.42 |
| Limestone (CaCO₃) | 0.75 | 0.75 | 0.69 |
| Salt (NaCl) | 0.36 | 0.37 | 0.34 |
| Choline Chloride 60% | 0.10 | 0.10 | 0.10 |
| Vitamin Mineral Premix | 0.50 | 0.50 | 0.50 |
| DL-Methionine | 0.25 | 0.20 | 0.21 |
| L-Lysine-HCl | 0.22 | 0.23 | 0.19 |
| L-Threonine | 0.04 | 0.04 | 0.03 |
| Total | 100 | 100 | 100 |
| Nutrient composition, as is | | | |
| ME, kcal/kg | 3008 | 3086 | 3186 |
| CP, % | 23.90 | 20.45 | 19.69 |
| Ca | 0.90 | 0.84 | 0.76 |
| Available Phosphorous | 0.45 | 0.42 | 0.38 |
| Lysine | 1.36 | 1.15 | 1.09 |
| Methionine | 0.62 | 0.53 | 0.52 |
| Methionine + Cysteine | 1.00 | 0.86 | 0.84 |
| Threonine | 0.9, | 0.80 | 0.76 |
| Tryptophan | 0.27 | 0.23 | 0.21 |
| Arginine | 1.50 | 1.30 | 1.18 |
| Isoleucine | 1.00 | 0.85 | 0.80 |
| Leucine | 2.19 | 1.98 | 1.91 |
| Valine | 1.10 | 0.95 | 0.90 |

Three physiologically healthy birds were euthanized each at days 3, 15 and 35 to excise spleen, intestinal (ileum) and muscle (pectoralis major) samples for DNA extraction (an Invitrogen PureLink genomic DNA isolation kit) and bisulfite sequencing.

### Samples

Animals were stratified into three tissue (breast, ileum and spleen) and three age (3d, 15d, 34d) groups. From each of these 9 groups, DNA was prepared from three independent animals, resulting in 27 genomic DNA samples.

### Whole Genome Bisulfite Sequencing

Whole-genome bisulfite sequencing services were conducted. Libraries were prepared using the Accel-NGS Methyl-Seq DNA Library Kit from Swift Biosciences. Two sequencing libraries were barcoded onto one sequencing lane. Sequencing was performed on an Illumina HiSeq X platform using a standard paired-end sequencing protocol with 105 nucleotides read length.

### Read Mapping

Reads were trimmed and mapped with BSMAP 2.5 (Xi Y, Li W. 2009. BSMAP: whole genome bisulfite sequence MAPping program. BMC Bioinformatics 10:232. doi:10.1186/1471-2105-10-232.) using the Gallus gallus genome assembly version 5.0 (https://www.ebi.ac.uk/ena/data/view/GCA_000002315.3) as a reference sequence. Duplicates were removed using the Picard tool (http://broadinstitute.github.io/picard). Methylation ratios were determined using a Python script (methratio.py) distributed together with the BSMAP package by dividing the number of reads having a methylated CpG at a certain genomic position by the number of all reads covering this position.

### Establishment of a Chicken DNA Methylation Clock

A penalized regression model (implemented in the R package glmnet [https://cran.r-project.org/web/packages/glmnet/]) was used to regress the chronological age on the CpG probes in the training set. For the genome-wide clock, we restricted the analysis to CpGs that showed a strand specific coverage of greater than 3 in every of the sequenced samples, resulting in a set of 12,876,934 CpGs. For the LMR clock, we restricted the analysis to CpGs within low-methylated regions that showed a strand specific coverage of greater than 3 in every of the sequenced samples, resulting in a set of 765,266 CpGs.

### Results

The alpha parameter of glmnet was varied in a range between 0 and 1 and chosen as 0.54 (elastic net regression), because this value led to a fit that was close to the best fit and a manageable amount of CpGs. The lambda value was chosen using cross-validation on the training data as 0.668. This identified a set of 63 CpGs together with corresponding beta values, which define the weights for these CpGs used in the chicken methylation clock. The mean squared error of 9-fold crossvalidation using the values of 0.54 for alpha and 0.668 for lambda was 2.912493 days. This indicates that a new sample can be predicted with an error of about 1.7 days. In order to apply the clock to a new sample the methylation ratios of this sample at the 63 clock CpGs have to be provided and the command predict.cv of the package glmnet with the trained clock has to be performed.

Table 2

Clock CpGs (genome-wide methylation, alpha = 0.54, lambda = 0.6688101, #CpG’s: 63).

| ID | chromosome | position of C (Gallus gallus genome assembly version 5.0) | Weighting factor of C in linear model equation, found by glmnet |
|---|---|---|---|
| 1 | chr1 | 25008830 | 0.1201470442 |
| 2 | chr1 | 25111167 | 1.9877222333 |
| 3 | chr1 | 25111203 | 0.8410870744 |
| 4 | chr1 | 25215851 | 1.2141924707 |
| 5 | chr1 | 25224869 | 0.0017297228 |
| 6 | chr1 | 38151254 | -1.1629356009 |
| 7 | chr1 | 98225254 | -0.6425407389 |
| 8 | chr1 | 98232924 | -0.0209456299 |
| 9 | chr1 | 98449317 | 2.4306655726 |
| 10 | chr1 | 120302870 | -0.5266391056 |
| 11 | chr1 | 122381098 | 0.2068133074 |
| 12 | chr1 | 136726442 | 0.1064898370 |
| 13 | chr1 | 136947007 | 0.7150939026 |
| 14 | chr1 | 136947024 | -0.1005845211 |
| 15 | chr1 | 155719172 | -0.3553818254 |
| 16 | chr1 | 165952547 | -1.0686961681 |
| 17 | chr2 | 37156274 | -0.7234326082 |
| 18 | chr2 | 50274933 | -1.9509594895 |
| 19 | chr2 | 65836919 | 0.0012483399 |
| 20 | chr2 | 84075642 | 0.0753794983 |
| 21 | chr2 | 97679432 | -0.8587247218 |
| 22 | chr2 | 124393300 | 0.0008574341 |
| 23 | chr2 | 130294650 | -0.2675000430 |
| 24 | chr3 | 37670532 | 0.2441000022 |
| 25 | chr4 | 17114627 | -0.0144030257 |
| 26 | chr4 | 17988283 | -0.8468332860 |
| 27 | chr4 | 30020074 | -0.8090270874 |
| 28 | chr4 | 56349569 | -0.2213015568 |
| 29 | chr4 | 83974212 | 1.3688948067 |
| 30 | chr5 | 12624758 | -0.5512961131 |
| 31 | chr5 | 41390309 | -0.0005570298 |
| 32 | chr5 | 41470627 | 0.4950833229 |
| 33 | chr5 | 43219792 | 0.0102589180 |
| 34 | chr5 | 43267010 | -0.5840711563 |
| 35 | chr5 | 43287871 | -0.9018947773 |
| 36 | chr5 | 52981453 | -1.6852256551 |
| 37 | chr5 | 52988321 | -0.0034053300 |
| 38 | chr5 | 54867941 | 0.9552754916 |
| 39 | chr6 | 10832725 | 0.7998053520 |
| 40 | chr8 | 21190795 | -0.3469761054 |
| 41 | chr8 | 24062272 | -0.0340524553 |
| 42 | chr8 | 27668980 | -0.0117602447 |
| 43 | chr10 | 9910081 | 0.8644423076 |
| 44 | chr10 | 9910314 | 0.3696403999 |
| 45 | chr10 | 9910365 | 0.5854942871 |
| 46 | chr10 | 10336823 | 0.3635723481 |
| 47 | chr10 | 10841972 | 0.3427303092 |
| 48 | chr12 | 11080808 | 0.8281174497 |
| 49 | chr12 | 13217820 | -0.6668570734 |
| 50 | chr13 | 10242508 | -0.0068685759 |
| 51 | chr14 | 5256102 | 0.4507668235 |
| 52 | chr14 | 7431894 | -1.2268127639 |
| 53 | chr15 | 1894201 | 0.0047490047 |
| 54 | chr15 | 7687079 | -2.2098760578 |
| 55 | chr15 | 9810477 | 0.3282647376 |
| 56 | chr17 | 1680784 | 0.5952569372 |
| 57 | chr17 | 4917324 | 0.0043603462 |
| 58 | chr17 | 4920172 | 0.1375606500 |
| 59 | chr18 | 1275141 | -0.3368529858 |
| 60 | chr21 | 5371614 | -0.2070140738 |
| 61 | chr23 | 1181275 | -0.4956393650 |
| 62 | chr24 | 3272445 | -0.6063586075 |
| 63 | chr24 | 3311935 | -1.6906353118 |

Intercept of linear model equation found by glmnet: 20.6479265656

Table 3

Clock CpGs (LMR methylation, alpha = 0.54, lambda = 0.610, #CpG’s: 54).

| ID | chromosome | position of C (Gallus gallus genome assembly version 5.0) | Weighting factor of C in linear model equation, found by glmnet |
|---|---|---|---|
| 1 | chr1 | 25248650 | 0.193774594770605 |
| 2 | chr1 | 40591436 | -0.568643213730932 |
| 3 | chr1 | 40591679 | -0.638478225996738 |
| 4 | chr1 | 63795100 | 0.318371926245549 |
| 5 | chr1 | 117783796 | 0.461005791871399 |
| 6 | chr1 | 123160777 | -0.577270474407336 |
| 7 | chr1 | 179633224 | -0.628500576957 |
| 8 | chr2 | 4113495 | -0.826267487270121 |
| 9 | chr2 | 7824662 | 1.64648733634692 |
| 10 | chr2 | 16989601 | -1.22334588133107 |
| 11 | chr2 | 44987393 | -3.44735070874419 |
| 12 | chr2 | 54405445 | -0.0214814175623492 |
| 13 | chr4 | 12748089 | 1.70526212493669 |
| 14 | chr4 | 20601950 | -2.0957909954246 |
| 15 | chr4 | 52871995 | 1.3114692051496 |
| 16 | chr4 | 73741292 | -1.12309056504113 |
| 17 | chr4 | 81578474 | 1.26147815810531 |
| 18 | chr4 | 84535022 | 4.3231052389549 |
| 19 | chr5 | 12620145 | -0.458731514322129 |
| 20 | chr5 | 30814076 | -0.698405329529931 |
| 21 | chr5 | 56027840 | -0.482584639740165 |
| 22 | chr6 | 10260287 | 0.880413359385883 |
| 23 | chr6 | 12120484 | -2.10440924485703 |
| 24 | chr6 | 26156764 | -0.270864342181805 |
| 25 | chr6 | 32055247 | -2.69265299854456 |
| 26 | chr6 | 33836727 | 1.15966877632569 |
| 27 | chr7 | 31520228 | 0 |
| 28 | chr8 | 22843445 | -1.14886729390649 |
| 29 | chr8 | 24062272 | -2.78258737459703 |
| 30 | chr8 | 25204975 | -0.664256989487943 |
| 31 | chr8 | 27279773 | -2.4140540423127 |
| 32 | chr9 | 23007215 | 1.52778880867768 |
| 33 | chr10 | 17028547 | -0.740108869993362 |
| 34 | chr10 | 19606919 | -2.61801343344312 |
| 35 | chr10 | 19898184 | 0 |
| 36 | chr11 | 8253242 | 0.920254116414776 |
| 37 | chr11 | 16879868 | -1.81033374964972 |
| 38 | chr11 | 18940226 | 0 |
| 39 | chr12 | 13161054 | 0 |
| 40 | chr12 | 14733821 | 2.90096891915326 |
| 41 | chr13 | 17346033 | 1.39473025721805 |
| 42 | chr13 | 17381809 | 0 |
| 43 | chr14 | 13938035 | -1.58848264991683 |
| 44 | chr15 | 3464122 | 0.722266869090579 |
| 45 | chr15 | 6016063 | -0.251682283409453 |
| 46 | chr15 | 6041329 | -1.30521623912729 |
| 47 | chr17 | 3046991 | -0.533407893149151 |
| 48 | chr18 | 4397723 | -1.46131922207642 |
| 49 | chr18 | 4397729 | -3.19274009960717 |
| 50 | chr18 | 6084926 | -2.47686681997711 |
| 51 | chr24 | 2956007 | -0.010441921766146 |
| 52 | chr28 | 3202682 | -2.22932163085087 |
| 53 | chrZ | 52701266 | 0.269214728598885 |
| 54 | chrZ | 56931170 | 0.22912910718348 |

Intercept of linear model equation found by glmnet: 27.1994178510791

1. A method of establishing a chicken methylation clock, the method comprising:
(a) determining a methylation ratio and a read coverage of genomic CpG sites of an age-correlated training sample of a specific chicken tissue;
(b) defining a set of CpG sites having reliable methylation ratios in all training samples of the determining (a) using a cutoff value; and
(c) performing a penalized regression using the methylation ratios of the defining (b) as input and the age correlated to the training sample as dependent variable, by applying a penalized regression model;
thereby obtaining a set of CpG sites with corresponding weighting factors and intercept of a linear model equation as parameters defining the chicken methylation clock.

2. The method according to claim 1, wherein the methylation ratio and the read coverage of the genomic CpG sites are determined in the determining (a) using bisulfite sequencing.

3. The method according to claim 1, wherein the age-correlated training sample of the specific chicken tissue used in the determining (a) is selected from the group consisting of gut tissue, muscle tissue, organ tissue and skin tissue.

4. The method according to claim 1 , wherein the coverage cutoff value defined in the defining (b) is 3.

5. The method according to claim 1, the method further comprising:
(d) optimizing a fit of the regression model such that the number of CpG sites is reduced to below 100.

6. The method according to claim 5, wherein for optimizing the fit of the regression model in the optimizing (d), applying ridge regression in combination with lasso regression.

7. The method according to claim 6, wherein the methods of ridge regression and lasso regression are balanced using an alpha value of between 0 and 1.

8. An in vitro method for predicting the chronological age of a chicken, the method comprising:
a) obtaining genomic chicken DNA from biological sample material deriving from a chicken subject or from a chicken population to be tested;
b) determining a methylation level for the CpG sites indicated in Table 2 or, alternatively, for the CpG sites indicated in Table 3, in the genomic chicken DNA;
c) comparing the methylation levels of the CpG sites in the genomic chicken DNA from the sample material to be tested with the methylation levels of the same CpG sites from an age-correlated reference sample material,
and deducing therefrom the chronological age of the subject or the population to be tested.

9. A method for predicting the chronological age of a chicken tissue sample material, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain weighted methylation ratios of those CpG sites,
(c) computing a sum over the weighted methylation ratios obtained in the determining (b) and adding a respective intercept of a linear model equation,
thus predicting the chronological age for the chicken tissue sample.

10. The method according to claim 9, wherein the tissue sample material deriving from the chicken subject or from the chicken population to be tested in the obtaining (a) is selected from the group consisting of gut tissue, muscle tissue, organ tissue and skin tissue.

11. The method according to claim 9 , wherein the tissue sample material deriving from the chicken subject or from the chicken population to be tested used in the obtaining (a) is gut tissue, preferably isolated from fecal sample material.

12. The method according to claim, wherein the methylation ratios for the CpG sites in the determining (b) were determined using bisulfite sequencing.

13. A method for detecting accelerated aging in a chicken tissue sample material, the method comprising:
(a) obtaining genomic DNA from the tissue sample material deriving from a chicken subject or from a chicken population to be tested,
(b) determining the methylation ratios for the CpG sites indicated in Table 2 or, alternatively, for the CpG sites indicated in Table 3, and multiplying same with their respective weighing factors to obtain the weighted methylation ratios of those CpG sites,
(c) computing a sum over weighted methylation ratios obtained in the determining (b) and adding a respective intercept of a linear model equation, thus predicting the age for the chicken tissue sample (epigenetic age) and
(d) comparing the predicted age of the computing (c) with the actual chronological age of the tissue sample,
wherein a predicted age higher than the chronological age is indicative of accelerated aging in the chicken tissue sample.
